(19) United States
(12) Patent Application Publication
(10) Pub. No.: US 2015/0238639 A1
(43) Pub. Date: Aug. 27, 2015

(54) Contrast Agent and Applications Thereof

(71) Applicants: National University of Singapore, Singapore (SG); University of California, Los Angeles, Los Angeles, CA (US)

(72) Inventors: Weixin HOU, Singapore (SG); Edward Kai Hua CHOW, Singapore (SG); Dean HO, Los Angeles, CA (US)

(21) Appl. No.: 14/630,289

(22) Filed: Feb. 24, 2015

(30) Foreign Application Priority Data
Feb. 25, 2014 (GB) 1403248.6

(51) Int. Cl. A61K 49/10; A61K 49/08

(52) U.S. Cl. CPC A61K 49/106; A61K 49/103; A61K 49/085

(57) ABSTRACT

A reagent comprising a nanodiamond particle linked to a paramagnetic ion for use as a contrast agent in magnetic resonance (MR) imaging in which T2-weighted magnetic images are obtained, and in particular in which both T1- and T2-weighted magnetic images are obtained, are described and claimed. Also claimed are novel reagents of this type, methods for their preparation and their use in diagnostics.

This utility patent application claims priority to British Application No. GB 1403248.6, filed on Feb. 25. 2014.

### TECHNICAL FIELD

The present invention relates to reagents useful as contrast agents in particular in magnetic resonance (MR) imaging, as well as compositions comprising these reagents, methods for preparing them and their use in diagnosis and therapy.

### BACKGROUND

Various imaging techniques including magnetic resonance imaging (MRI), computed tomography (CT), positron emission tomography (PET), and optical microscopy in the bio-imaging fields, have been widely employed to increase the accuracy of disease diagnosis, especially for diseases such as cancer. Among these imaging methods, MR imaging is believed to be one of the most powerful diagnostic tools due to its inherent advantages such as non-invasiveness, safety, and high spatial resolution.

The use of contrast agents in magnetic resonance (MR) imaging facilitates a more accurate diagnosis by enhancing the contrast between tissues. For example, chelated complexes of paramagnetic metal ions such as Gd³⁺ and Mn²⁺ have a marked effect on spin-lattice relaxation of surrounding water protons and lead to bright contrast enhancement in T1-weighted MR imaging (positive contrast effect).

In addition, superparamagnetic iron oxide (SPIO) nanoparticles, which induce the dark contrast enhancement in T2-weighted MR images, based upon spin-spin lattice relaxation, have been commercialized as T2 contrast agents and are clinically applied in liver imaging. However, each mode contrast agent has its own unique advantages and limitations. For example, some clinical Gd based contrast agents may result in potential danger such as nephrogenic systemic fibrosis (NSF) for patients with severe renal disease or following liver transplant, as claimed by the Food and Drug Administration. (FDA) organization since 2006. On the other hand, the clinical applications of iron oxide based contrast agents are quite limited because of magnetic susceptibility artefacts and their negative contrast effect, which may not be clearly distinguishable from the low level MR signal arising from adjacent tissues such as bone or vasculature.

Therefore, there is a need for dual-mode contrast agents (DMCAs) which allow a combination of two different modes of imaging (T1- and T2-weighted MR imaging) to be taken in order to improve the diagnosis accuracy of diseases. The greatest advantage of the dual imaging strategies is that two complementary images can be provided simultaneously by employing a single instrumental system, compared with other bimodal imaging technologies (e.g., MR/optical), which need to consider the different penetration depths and spatial time resolutions of multiple imaging devices.

Unfortunately, most of the reported DMCAs usually consisted of two kinds of functional species: one is commonly Gd- or Mn-based material for T1-weighted MR Imaging; the other is Fe-based nanoparticles for T2-weighted MR imaging. Due to the inevitable severe interference between these two different contrast agents, it is difficult to develop high quality DMCAs with simultaneously high T1 relaxivity and T2 relaxivity. Thus, developing a class of novel DMCAs with single component (Fe-, Mn-, and Gd-based) without conflicting effects between the two kinds of functional units is still a great challenge.

Nanodiamond, a carbon-based nanoparticle has aroused great interest due to its excellent biocompatibility. It has been used in a variety of biological and non-biological applications. For instance, the use of nanodiamond conjugated to chemotherapeutic moieties to treat certain cancers has been described (E. Chow et al., Sci. Transl. Med. 2011, 73, p 73ra21). In addition, anazide-modified nanodiamond carrying a photoacitvatable CO releasing molecule based a manganese carbonyl complex has been described (Dördelmann G. et al. (2012) Chem. Comm. 2, 48 m 11528-11530).

The surface of nanodiamond can absorb water molecules and be modified easily, which means that nanodiamond is a potential platform as a MR imaging contrast agent. This was particularly well demonstrated with the conjugation of Gd and enhanced relaxivity results in T1 weighted imaging (Manus et al. J. Nano Lett. 2010, 10, 484-489).

The applicants have found however that such agents and in particular, certain novel agents based upon manganese Mn²⁺ ions are useful in T2 as well as T1 weighted imaging and thus can be used as effective dual contrast agents.

### SUMMARY

According to a first aspect, the present invention provides a reagent comprising a nanodiamond particle linked to a paramagnetic ion for use as a contrast agent in magnetic resonance (MR) imaging in which T2-weighted magnetic images are obtained.

In particular, the reagent is for use as a dual-mode contrast agent (DMCA) in magnetic resonance (MR) imaging, in which both T1 and T2 weighted magnetic images are obtained.

Reagents of this type represent a new class of dual-mode contrast agents for MR imaging. Compared to single mode MR imaging contrast agent, these agents have significant advantages in that they allow the production of a combined T1/T2 weighted MR images, which leads to improvements in diagnosis accuracy of diseases.

In contrast to many other reported DMCAs, the reagents of the present invention are single component DMCAs and thus avoid interference that may be caused when two different contrast agents are used. Furthermore, they have shown no observable adverse effect level (NOAEL) in maximum tolerable dose (MTD) studies and appear to be of low toxicity. In particular, they are of lower toxicity than some conventional contrast agents, such as manganese chloride.

Suitable paramagnetic ions for use in the reagents of the invention include those conventionally used in MR imaging including Mn²⁺, Gd³⁺, Eu³⁺, Tm³⁺, Yb³⁺ and Fe³⁺. In particular, the paramagnetic ion is Mn²⁺ or Gd³⁺ and in a particular embodiment is Mn²⁺.

Suitable nanodiamond particles for use in the reagents of the invention are available commercially. They are generally obtained by detonation of carbon based explosive materials. Particles are generally less than 10 nm in diameter, for example from 2-8 nm in diameter.

The paramagnetic ion can be linked to the nanodiamond particle in a variety of ways. For instance, the paramagnetic ion can be covalently attached to the nanodiamond particle by way of a linking grouper they may be co-ordinated or conjugated together.

In a particular, embodiment, the paramagnetic ion is attached to the nanodiamond surface by way of an organic linking group. Such groups may suitably contain from 2-100 atoms, preferably from 10-50 atoms which are selected from carbon atoms and heteroatoms such as, but not limited to, nitrogen, oxygen, silicon and sulphur atoms. The atoms are suitably arranged in straight or branched chains. The length of the chain is of the organic linking group impacts on the values of r1/r2 that can be obtained in an MR imaging process. Increasing the length of the organic linking group may lead to enhancement in the values.

In a particular embodiment, the linking group includes a chelating moiety, which sequesters the paramagnetic ion. Examples of such chelating moieties include derivatives of citric acid, diethylenetriaminepentaacetic acid (DTPA), ethylene-diaminetetraaceticacid (EDTA), 1,4,7,10-tetraazacyclododecane-N,N′,N″,N′″-tetraacetic acid (DOTA), 1,4,7,10-tetraazacyclododecane-N,N′,N″-triacetic acid, 3,6,9-triaza-12-oxa-3,6,9-tricarboxymethylene-10-carboxy-13-phenyl-tridecanoic acid (B-19036), hydroxybenZylethylenediaminediacetic acid (HBED), N,N′-bis(pyridoxyl-5-phosphate)ethylene diamine, N,N′-diacetate (DPDP), 1,4,7-triazacyclononane-N,N′,N″ triacetic acid (NOTA), 1,4,8,11-tetraaZacyclotetradecane-N,N′,N″,N′″-tetraacetic acid (TETA), kryptands (macrocyclic complexes), and desferrioxamine, as well as anhydrides, dianhydrides or esters thereof.

Additionally, the linking group may comprise surface activating moieties which are linked directly to the surface of the diamond nanoparticle and also to the chelating groups as described above. In particular, the surface activating moieties may include free functional groups, such as amino or carboxyl groups, that can bind to the chelating moieties as described above. These free functional groups may be attached via hydrocarbyl groups for example containing from 1-10 carbon atoms, to binding groups which will become attached to the surface of the nanodiamond. Suitable hydrocarbyl groups include alkyl, alkenyl, alkynyl or aryl groups such as phenyl groups. In particular, the free functional groups such as amino groups are linked to the binding groups by hydrocarbyl groups selected from C₁₋₆alkyl groups such as propyl or butyl or phenyl groups.

Particular examples of binding groups that link to the surface of the diamond nanoparticle are alkoxysilanes, such as C₁₋₆alkoxysilanes. Suitably, each silane group carries from 1-3 alkoxy groups. In particular, the binding groups are trimethoxy or triethoxysilanes.

The reagents may be prepared by various procedures.

In one embodiment, they are prepared by reacting the paramagnetic ion, for example in the form of a salt such as a halide salt, carbonate, bicarbonate, sulphate of bisulphate salt, with a nanodiamond particle which carries a chelating group that forms a conjugate with said paramagnetic ion.

In this instance, the chelating agent is suitably linked to surface activating groups present on the surface of the diamond nanoparticle in a preliminary step. Suitable chelating agents are as described above, and include EDTA, citric acid, DTPA, DOTA, 1,4,7,10-tetraaZacyclododecane-N,N′,N″-triacetic acid, B-19036, HBED, N,N′-bis(pyridoxyl-5-phosphate)ethylene diamine, DPDP, NOTA, TETA, kryptands and desferrioxamine as well as anhydride or dianhydride derivatives thereof. A particular example of a chelating agent is EDTA or the anhydride or dianhydride thereof. The reaction is suitably carried out in a liquid suspension, where the liquid is for example a sodium bicarbonate solution, under an inert atmosphere, for example of an inert gas such as Argon. Sonication may optionally be applied during the reaction.

In this case also, the nanodiamond particle is suitably activated, in a preliminary step, for instance by reaction with a surface activating agent as described, above. Particular examples of surface activating agents include aminoalkyl or aminoaryl-alkoxysilanes such as (3-aminopropyl)-trimethoxysilane, 3-aminopropyltriethoxysilane, 4-aminobutyltriethoxysilane, and aminophenyltrimethoxysilane.

The activation reaction of the preliminary step is suitably carried out in suspension in a liquid such as a mixture of water and an alcohol such as ethanol. A reduced pH, for example of from 4.0-6.5 such as between 4.5-5.5 is suitably employed.

Alternatively, the reagents may be prepared by reacting a nanodiamond particle with a chelate comprising said paramagnetic ion wherein said chelate is able to link to said nanodiamond particle or a functional group present on the surface of the nanodiamond particle.

Suitable chelating agents are as described above. These may be reacted with the paramagnetic ion, for example in salt form as described above, in a preliminary step. The resultant chelate may then be linked to a nanodiamond particular whose surface has previously been activated as described above.

Alternatively, a chelating agent which is able to directly conjugate the paramagnetic ion to the surface of the nanodiamond particle is used. An example of such a chelating agent is N-(trimethoxysilylpropyl)ethylenediaminetriacetate. In such cases, there may be no need to activate, the surface of the nanodiamond particles.

Reagent may be isolated from the resultant reaction mixtures by techniques such as filtration, centrifugation etc. It may be washed before being prepared for use as a contrast agent in MR imaging. In particular, the reagent is isolated using repeated centrifugation/wash cycles, for example from 2-5 centrifugation/wash cycles, such as about 3 centrifugation/wash cycles.

The relative amounts of the reagents used in the reaction will depend upon factors such as the specific nature of those reagents, the required concentration of paramagnetic ions on the particles and the scale of the manufacture. Suitably however, an excess of reagents (surface activating agents, chelating agents, paramagnetic ions) are contacted with nanodiamond particles to ensure that maximal surface activation and chelation occurs.

Certain reagents as described above are novel and form a further aspect of the invention.

In a second aspect of the invention there is provided a dual-mode contrast agent comprising nanodiamond linked to a paramagnetic ion selected from Mn²⁺, Eu³⁺, Tm³⁺, Yb³⁺ and Fe³⁺.

In a particular embodiment, the paramagnetic ion is Mn³⁺. Suitably, the dual-mode contrast agent comprises a Mn²⁺ ion containing compound which is conjugated to nanodiamond particles (ND-Mn).

These reagents may be prepared using the methods described above.

Thus, in a third aspect, the invention provides a method for preparing a dual-mode contrast agent comprising nanodiamond particles linked to a paramagnetic ion selected from Mn²⁺, Eu³⁺, Tm³⁺, Yb³⁺ and Fe³⁺, said method comprising either
(A) reacting the paramagnetic ion with a nanodiamond particle which carries chelating group that forms a conjugate with said paramagnetic ion; or
(B) reacting a nanodiamond particle with a chelate comprising said paramagnetic ion wherein said chelate is able to link to said nanodiamond particle or a functional group present on the surface of the nanodiamond particle; and
recovering dual mode contrast agent from reaction mixtures formed.

In the case of option (A) above, the paramagnetic ion used in the reaction is suitably in the form of salt, such as a halide salt (for instance a fluoride, chloride, bromide or iodide salt), a carbonate, bicarbonate, sulphate, bisulphate or salt. In particular, the paramagnetic ion is manganese and the salt is a manganese chloride. This is contacted with a nanodiamond particle which carries a chelating group that forms a conjugate with said paramagnetic ion, suitably in solution.

Nanodiamond particles carrying the chelating groups are suitably prepared by first activating a nanodiamond particle as described above, so that is carries a free functional group such as amino or carboxyl groups, and in particular free amino groups. Particular examples of surface activating agents useful in this reaction include aminoalkyl or aminoaryl-alkoxysilanes such as (3-aminopropyl)-trimethoxysilane, 3-aminopropyltriethoxysilane, 4-aminobutyltriethoxysilane, and aminophenyltrimethoxysilane. As described above, the activation reaction is suitably carried out in suspension in a liquid such as a mixture of water and an alcohol such as ethanol. A reduced pH, for example of from 4.5-6.5 such as about 5.5 is suitably employed.

The activated nanodiamond may then be reacted with a chelating agent, also as described above. Suitable chelating agents are as described above, and include EDTA, citric acid, DTPA, DOTA, 1,4,7,10-tetraaZacyclododecane-N,N′,N″-triacetic acid, B-19036, HBED, N,N′-bis(pyridoxyl-5-phosphate)ethylene diamine, DPDP, NOTA, TETA, kryptands and desferrioxamine as well as anhydride, or dianhydride derivatives thereof. A particular example of a chelating agent is EDTA or the anhydride or dianhydride thereof. The reaction is suitably carried out in a liquid suspension, where the liquid is for example a sodium bicarbonate solution, under an inert atmosphere, for example of an inert gas such as Argon. Sonication may optionally be applied during the reaction.

In an alternative embodiment, the nanodiamond may react directly with the chelating agent without prior activation, for example where the chelating agent is a dihydride such as EDTA dianhydride. In these instances, nanodiamond particles may be mixed with solutions of the chelating agent in a suitable organic solvent, such as dimethylformamide (DMF). The mixture may be heated and/or sonicated for a period sufficient to ensure that the chelating agent becomes associated with the nanodiamond surface.

Alternatively, the reagents may be prepared by contacting a nanodiamond particle with a chelate comprising said paramagnetic ion wherein said chelate is able to link to said nanodiamond particle or a functional group present on the surface of the nanodiamond particle, as described above.

Reagents of the invention are suitably formulated together with pharmaceutically acceptable carriers for administration to patients.

Thus a fourth aspect of the invention provides a pharmaceutical composition comprising a reagent as described above, and in particular a reagent of the third aspect of the invention in combination with a pharmaceutically acceptable carrier.

Suitable pharmaceutical compositions will be in either solid or liquid form. They may be adapted for administration by any convenient peripheral route, such as parenteral or oral administration or for administration by inhalation or insufflation. The pharmaceutical acceptable carrier may include diluents or excipients which are physiologically tolerable and compatible with the active ingredient. These include those described for example in Remington's Pharmaceutical Sciences, Mack Publishing Co. (A. R. Gennaro edit, 1985).

Parenteral compositions are prepared for injection, for example by either subcutaneous, intramuscular, intradermal, intravenous, intraperitonea, intraosseous, epidural, intracardiac, intraarticular, intracaverous or interavitreal injection or via needle-free injection systems. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

The amount of agent administered will vary depending upon factors such as the specific nature of the reagent used, the size and health of the patient, the nature of the condition being diagnosed etc. in accordance with normal clinical practice. Typically, a dosage in the range of from 0.01-1000 mmol/Kg, for instance from 0.1-10 mmol/Kg, would produce a suitable imaging properties. Dosages may be given in single dose regimes, split dose regimes and/or in multiple dose regimes lasting over several days, depending upon the MR strategy adopted.

The reagent of the present invention may be used in combination with one or more other active agents, such as one or more other imaging reagents or pharmaceutically active agents. Examples of such active agents include proteins, peptides, small molecules, genetic material, other biological material and other imaging reagents for the purpose of dual-therapeutics and diagnostics as well as targeted imaging complex delivery.

These additional active agents may be administered simultaneously or sequentially with the reagents of the invention. Where they are administered simultaneously, they may be combined with the reagent of the invention in a single pharmaceutical composition as described above. In some instances, the additional active agent may also be conjugated to the nanodiamond particle of the reagents of the invention.

As described above, reagents of the invention may be used in MR imaging since they showed excellent relaxivity results in both T1 and T2 weighted imaging. In particular, they show a change particular a reduction) T1/T2 relaxation time resulting in altered (e.g. increased) signal intensity on T1/T2 weighted images. This allows for improved imaging and thus more accurate diagnosis.

A fifth aspect of the invention provides a method for obtaining a magnetic resonance image of a cell, tissue, organ, or subject, said method comprising administering to said cell, tissue, organ, or subject, a reagent as described above, and subjecting said cell, tissue, organ, or subject to an MRI procedure to image said tissue. Since the reagents of the invention showed excellent relaxivity results in both T1 and T2 weighted imaging, both a T1 and 12 determination may be made. These results may be assessed for example to determine the T1/T2 or T2/T1 ratio(r). The results obtained may be used in methods of diagnosing disease, and these form a sixth aspect of the invention.

In particular the cells, tissues, organs, or subjects are eukaryotic or prokaryotic. In a particular embodiment, the subject is a human or non-human animal in particular a mammal. Suitable mammals include canines, porcines, equines, rodents such as rats or mice, bovines, felines, non-human primates, or humans. In particular the subjects are humans. In a particular embodiment, the reagents as described above and in particular Mn²⁺/nanodiamond conjugates are administered to the cell or subject and an MR image of at least a part of the cell or subject to which the conjugate has distributed is obtained.

Known methods for administering therapeutics and diagnostics can be used to administer reagents as described above. These methods include vacularly or parenterally for example by subcutaneous, intramuscular, intravenous, intradermal, intraperitoneal, intraosseous, epidural, intracardiac, intraarticular, intracaverous or interavitreal injection. Alternatively, they may be administered orally via the gastrointestinal tract, or by inhalation or insufflation.

Reagents as described above may be included in kits supplied for use in conjunction with MRI procedures. Thus a seventh aspect of the invention comprises a reagent as described above together with an additional element required in MRI procedures. These may include for example elements such as syringes, connectors and valves that may be useful in the administration of the reagent to allow it to be used as a contrast agent in an MRI procedure. The reagent may be in a dried form for reconstitution on site, or it may be in the form of a sterile suspension.

### BRIEF DESCRIPTION OF THE DRAWING FIGURES

The examples reference the accompanying diagrammatic drawings in which:

FIG. 1 shows a scheme for synthesis of a reagent of the invention which is a nanodiamond-manganese conjugate, involving modifying the nanodiamond surface with EDTA;

FIG. 2 shows FTIR spectra of (a) nanodiamond; (b) nanodiamond modified with EDTA;

FIG. 3 shows MR T1 (a)/T2 (b) images of nanodiamond samples. 1: water; 2: 15 μM Mn(II); 3: 30 μM Mn(II); 4: 60 μM Mn(II);

FIG. 4 shows graphs of relaxivity values of (a) r1 and (b) r2 obtained from the slopes of linear fits of experimental data for an EDTA modified nanodiamond complex;

FIG. 5 shows FTIR spectra of (a) nanodiamond; (b) nanodiamond modified with EDTA, prepared using an alternative route;

FIG. 6 shows graphs of relaxivity values of (a) r1 and (b) r2 obtained from the slopes of linear fits of experimental data for an EDTA modified nanodiamond complex of FIG. 5;

FIG. 7 shows FTIR spectra of (a) nanodiamond; (b) nanodiamond modified with DOTA;

FIG. 8 shows graphs of relaxivity values of (a) r1 and (h) r2 obtained from the slopes as of linear fits of experimental data for a DOTA modified nanodiamond complex;

FIG. 9 shows MRI images of mice liver (a) before MnCl₂ injection; (b) after MnCl₂ injection; (c) before ND-DOTA . . . Mn injection and (d) after ND-DOTA . . . Mn injection;

FIG. 10 shows T1-weighted MRI inniges of mice liver (a) before MnCl₂ injection; (b) after MnCl₂ injection; (c) before ND-DOTA . . . Mn injection and (d) after ND-DOTA . . . Mn injection;

FIG. 11 shows T2-weighted MRI images of mice liver (a) before MnCl₂ injection; (b) after MnCl₂ injection; (c) before ND-DOTA . . . Mn injection and (d) after ND-DOTA . . . Mn injection; and

FIG. 12 is a graph showing the results of a comparative toxicity study, carried out using THLE-2 immortalised hepatocytes, where, on the x axis, ND represents nanodiamond, Mn represents manganese chloride, ND-Mn represents nanodiamond linked to EDTA which has been chelated to manganese and the ND-NH2-Mn is functionalized nanodiamond linked to manganese by way of a DOTA chelator, the y axis shows growth inhibition/viability by MTT assay as normalized to untreated THLE-2 controls.

### DETAILED DESCRIPTION

The invention will now be particularly described by way of example. However, it will be apparent to one skilled in the art that the specific details are not required in order to practice the invention. The following descriptions of specific embodiments of the present invention are presented for purposes of illustration and description. They are not intended to be exhaustive of or to limit the invention to the precise forms disclosed. Many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated.

### EXAMPLE 1

### Preparation and Characterisation of ND-EDTA . . . Mn Contrast Agent

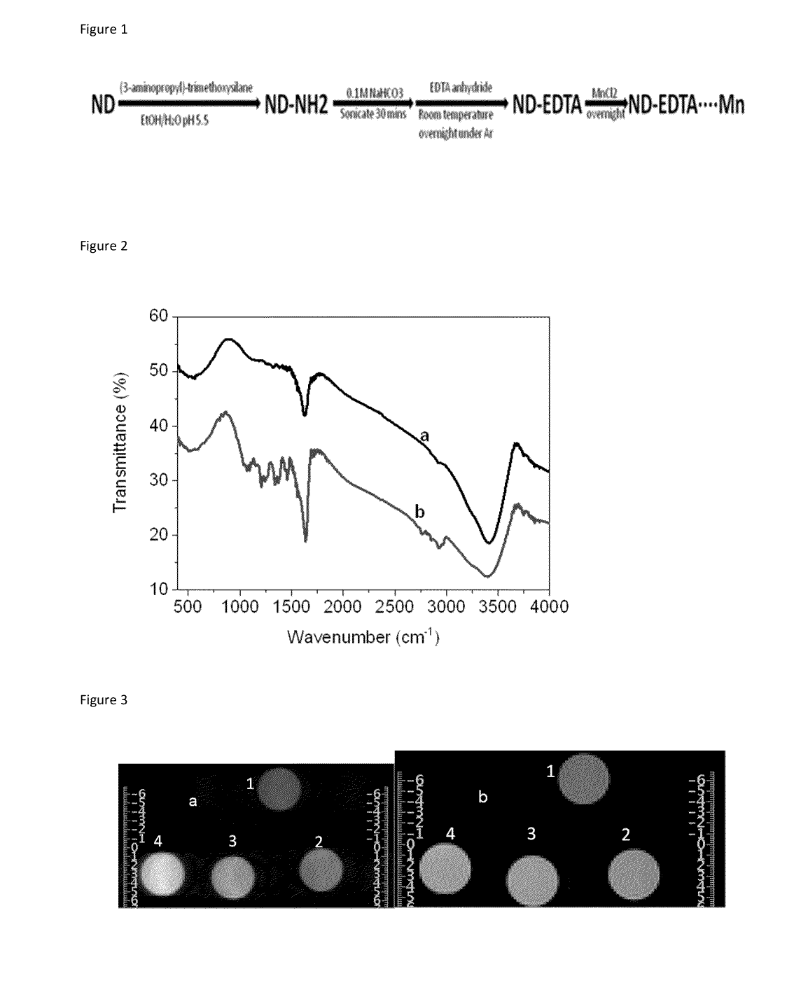

A contrast agent of the invention was prepared as illustrated schematically in FIG. 1.

Specifically, an amine-functionalized nanodiamond (ND-NH₂) was produced by adding (3-aminopropyl)-trimethoxysilane (2.5 g)) to a suspension of nanodiamonds (ND) (1.5 g) having a particle size in the range of 20-80 nm in a mixture of ethanol (47.5 ml) and water (2.5 ml). Acetic acid was added to the resultant mixture to adjust the pH to with the range of 4.5-5.5. The mixture was maintained at ambient temperature for 30 minutes.

Then, under an inert atmosphere (Argon), 30 mg of the ND-NH₂ obtained was dispersed in 0.1M NaHCO₃ solution (3 ml) and ethylenediaminetetraacetic (EDTA) dianhydride (22 mg) added. The mixture was sonicated for 30 minutes and maintained at room temperature overnight. Fourier transform infra-red spectroscopy (FTIR) results showed that the EDTA was successfully attached on the ND surfaces, as some of the EDTA characteristic peaks appeared on the ND surface as shown in FIG. 2.

The resultant ND-EDTA complex was chelated with Mn²⁺ by mixing with manganese chloride (MnCl₂) (10 mM)and leaving the mixture to stand overnight. A nanodiamond-manganese (ND-Mn) complex was isolated from the reaction mixture after 3 centrifuge/wash cycles.

Inductively coupled plasma mass spectrometry (ICP-MS) analysis of the complex showed that about 0.02 μmol Mn²⁺ was attached on 1 mg ND surfaces.

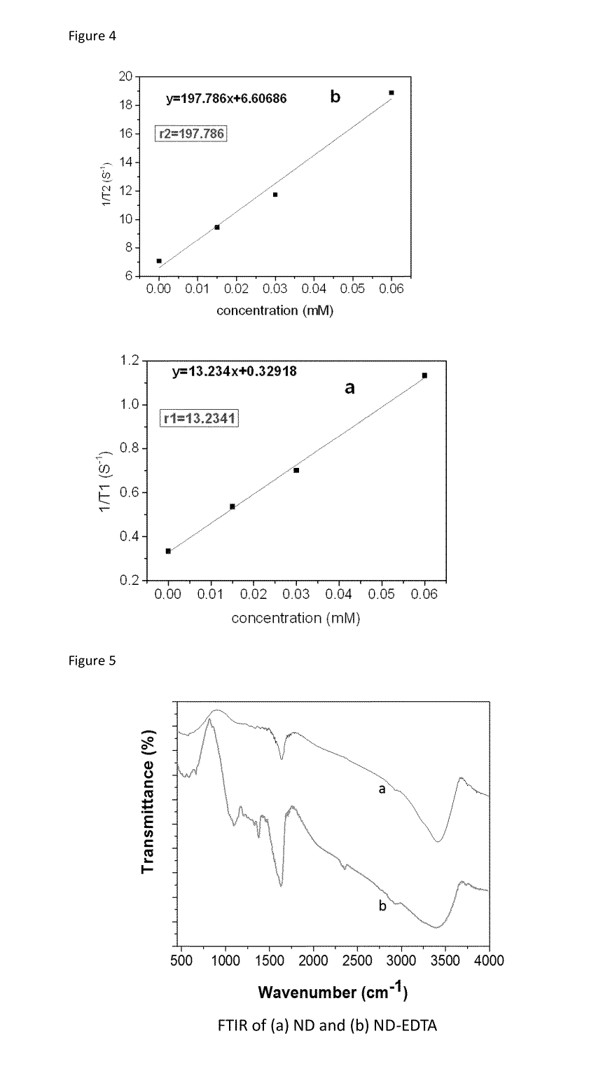

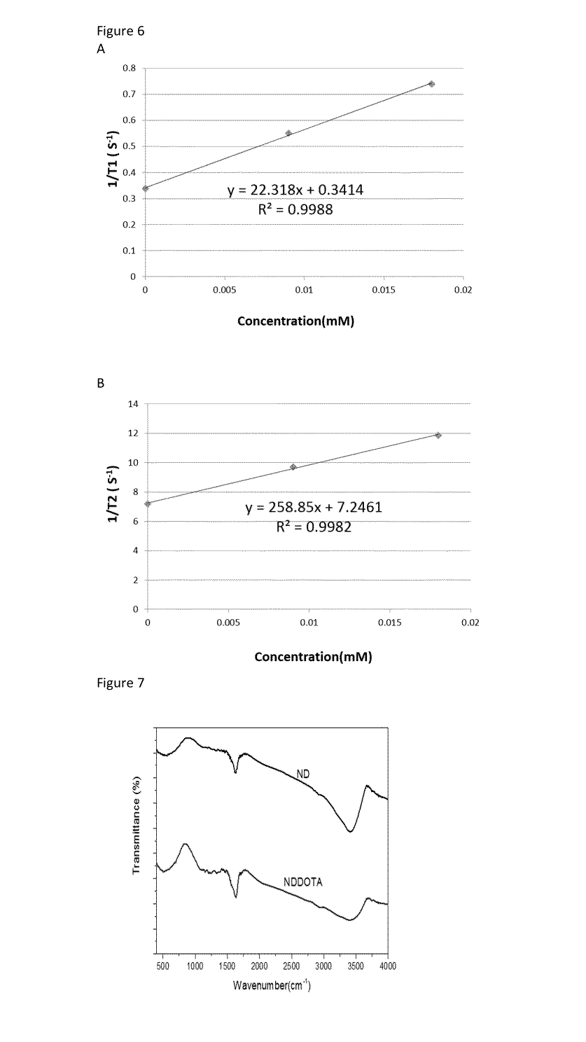

The resultant ND-Mn complex was dispersed in water at different Mn²⁺ concentrations (15 μM, 30 μM and 60 μM), and the samples were imaged on a 3T MRI seamier. Both T1-weighted images and T2-weighted images were obtained. The results for each of the different Mn²⁺ concentrations are shown in FIG. 3. Brightening of T1-weighted images and darkening of T2-weighted images demonstrates dual contrast enhancement to by the ND-Mn complex.

The results also showed in the following tables.

| sample | [Mn](mM) | T1(ms) |
|---|---|---|
| water | 0 | 2987 |
| ND-Mn | 0.015 | 1884 |
| ND-Mn | 0.03 | 1426 |
| ND-Mn | 0.06 | 882.3 |

| sample | [Mn](mM) | T2(ms) |
|---|---|---|
| water | 0 | 141.2 |
| ND-Mn | 0.015 | 105.7 |
| ND-Mn | 0.03 | 85.04 |
| ND-Mn | 0.06 | 52.93 |

Compared to pure water, both T1 and T2 results are decreased upon the addition of the ND-Mn.

To quantitatively evaluate the MR contrast enhancements, the longitudinal (r1) and transverse (r2) relaxivity values were calculated through the curve fitting of relaxation time versus the metal concentration (FIG. 4). The results showed that the r1 and r2 values of ND-Mn are 13.2 and 197.7 mM⁻¹s⁻¹, respectively. This is significantly higher than other reported Mn-based DMCAs. For instance, it is higher than the r1 value of 8.26 mM⁻¹s⁻¹ reported by Tian et al., Sci Rep. 2013 Dec. 5; 3:3424. doi: 10.1038/srep03424 obtained using manganese oxide nanoparticles.

### EXAMPLE 2

### Alternative Preparation and Characterisation of ND-EDTA . . . Mn Contrast Agent

Without amination the nanodiamond (ND) surface, the ND can also react with EDTA dianhydride directly. NanoAmando Soft Hydrogel was freeze-dried to obtain nanodiamond (ND) powder. EDTA dianhydride (100 mg) was heated to dissolve in 10 ml of dimethylformamide (DMF). ND powder (100 mg) was sonicated in DMF (10 ml) for 30 minutes, then added to the EDTA dianhydride solution. The mixture was reacted at 80° C. for 4 hours to get ND-EDTA. The as-prepared ND-EDTA powder was dispersed in water at 10 mg/ml and treated with 10 mM MnCl2 overnight. The ND-EDTA . . . Mn was washed with water until no manganese was detected form the supernatant.

FTIR showed that the EDTA is successfully modified on the ND surface, ICP results showed that around 0.0036 μmol Mn2+ was loaded on 1 mg ND. The zeta-potential of this complex is 35.2±2.5 mV, and diameter is 65.3+2.4311111. MRI test showed that the r1 and r2 values of ND-Mn in this ease were also high, at 22.318 and 258.85 mM⁻¹s⁻¹, respectively.

### EXAMPLE 3

### Preparation and Characterisation of ND-DOTA . . . Mn Contrast Agent

Similar methodology to that described in Example 1 was used to prepare a ND-DOTA-Mn contrast agent.

NanoAmando Soft Hydrogel (Nagno, Japan) was freeze-dried to obtain nanodiamond (ND) powder. Ethanol (EtOH) and H₂O were mixed together (95%/5%), then acetic acid (HAc) (1M) was added to the solution to adjust pH to 4.5-5.5.

(3-Aminopropyl)trimethoxysilane (APTMS) was added to the mixture to yield a concentration of about 5%. 5-10 minutes was allowed for the hydrolysis and silanol formation. ND was added to the solution and keeps stirring for another 2 hours. The product was centrifuged, and further washed with water. The centrifuge/wash cycles were performed at least 5 times. Then the product was freeze dried to get the NDNH₂ powder.

The NDNH₂ powder (100 mg) was sonicated in 0.1M NaHCO₃ for 30 minutes. DOTA-NHS (Macrocyclics) (20 mg) was dissolved in DMF (2 ml). These were then mixed together to react at room temperature with shaking overnight. The product was centrifuged, and further washed with water. The centrifuge/wash cycles were performed at least 5 times to get a DOTA functionalised nanodiamond complex (ND-DOTA).

Fourier transform infra-red spectroscopy (FTIR) results showed that the DOTA was successfully attached on the ND surfaces, as some of the. DOTA characteristic, peaks appeared on the ND surface as shown in FIG. 7.

ND-DOTA (50 mg) obtained was dispersed in water at a concentration of 10 mg/ml, then 1M MnCl₂ (100 μl) was added to the mixture and shaken overnight. The as-prepared ND-DOTA . . . Mn complex was washed with water until no Mn²⁺ was detected from the supernatant.

Hydrodynamic size and ζ-potential measurements were performed on a ZetasizerNano (Malvern, UK). Final values were averages of three or more separate measurements of each sample. Fourier transform infrared spectroscopy (FTIR) was performed using a Perkin-Elmer FTIR spectrum 2000 over a range of 400-4000 cm⁻¹. Samples were dried using a rotary evaporator. 5 mg of sample was mixed with 0.1 g KBr powder using mortar and pestle before pressing the sample to a thin film of which the spectra were taken. The resultant zeta-potential was 45.3±5.4 mV with a diameter of 75.2±8.26 nm.

Inductively coupled plasma mass spectrometry (ICP-MS) was used to determine the loading efficiency of Mn²⁺ on the nanodiamond. Samples (ND-DOTA . . . Mn) for analysis were digested with 37% HCl overnight. The sample was washed with water at least 5 times, and the supernatant was collected for the ICP-MS test. This analysis showed that in the complex, about 0.02±0.004 μmol Mn²⁺ was attached on 1 mg ND surfaces. Mn²⁺ standard with different concentrations were also tested at the same conditions.

In vitro relaxivity of theses complexes was also investigated. T1/T2 measurements were performed on a 7T Bruker MRI. A range of ND-DOTA . . . Mn with different Mn²⁺ concentrations were prepared for MRI phantom and relaxivity studies. The longitudinal relaxation times (T₁) were measured using an inversion recovery sequence, and transverse relaxation times (T₂) were measured using multi-echo multi-slice sequence. The longitudinal or transverse relaxivity (r₁ or r₂) was determined from the slope of the plot of 1/T₁ or 1/T₂ against the Mn²⁺ concentration.

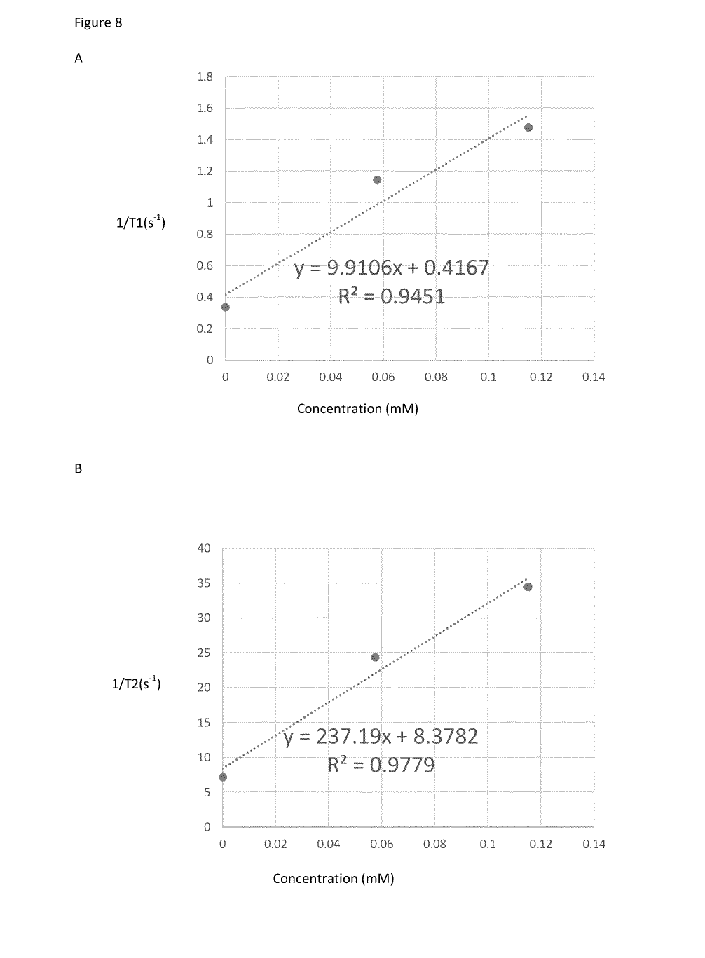

The results are shown in FIG. 8. The results showed that the r1 and r2 values of ND-Mn in this case were also high, at 9.9106 and 237.19 mM⁻¹s⁻¹, respectively.

### EXAMPLE 4

### In Vivo MR Imaging Studies

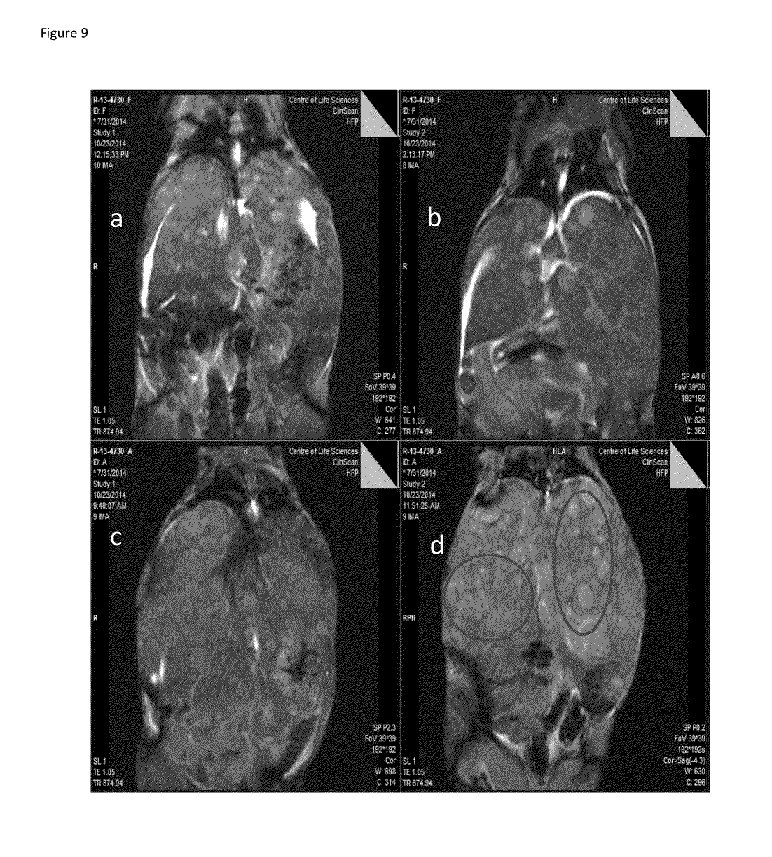

In vivo MR imaging was performed on a 7T Broker MRI using a fast spin-echo sequence. Different mice with average weights around 25 g bearing liver tumours were used for the experiments. MR images were taken prior to injection of samples using a known MRI agent, MnCl₂ (Contrast Media Mol. Imaging 2009, 4, 89-100) and the complex of Example 2 above. The MnCl₂ and ND-DOTA . . . Mn with Mn²⁺ content is around 0.1 μmol were injected through the tail vein. Conventional MRI images were taken (FIG. 9). Using this method, the tumours were most obvious in panel (d), after ND-DOTA . . . Mn injection.

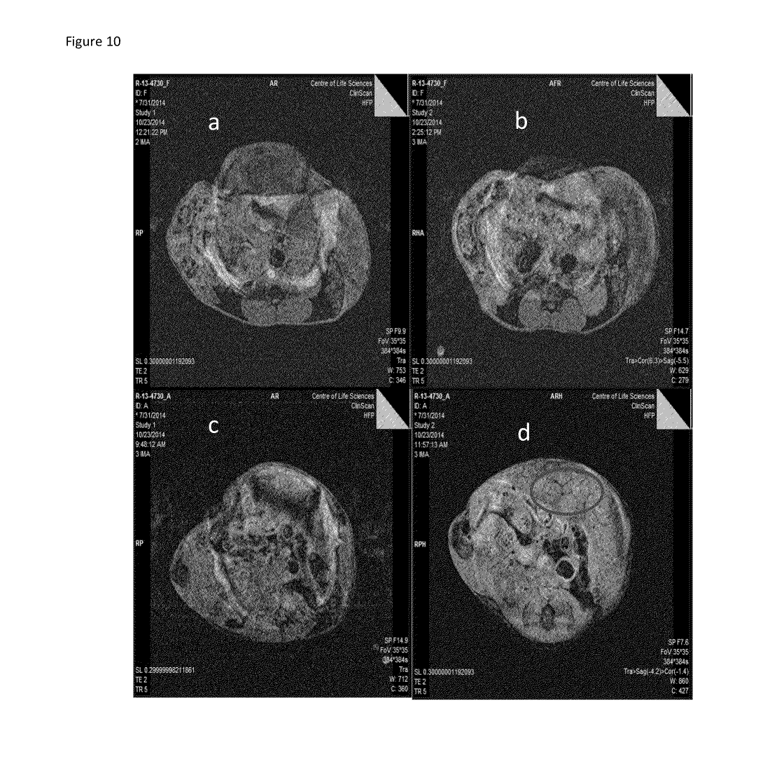

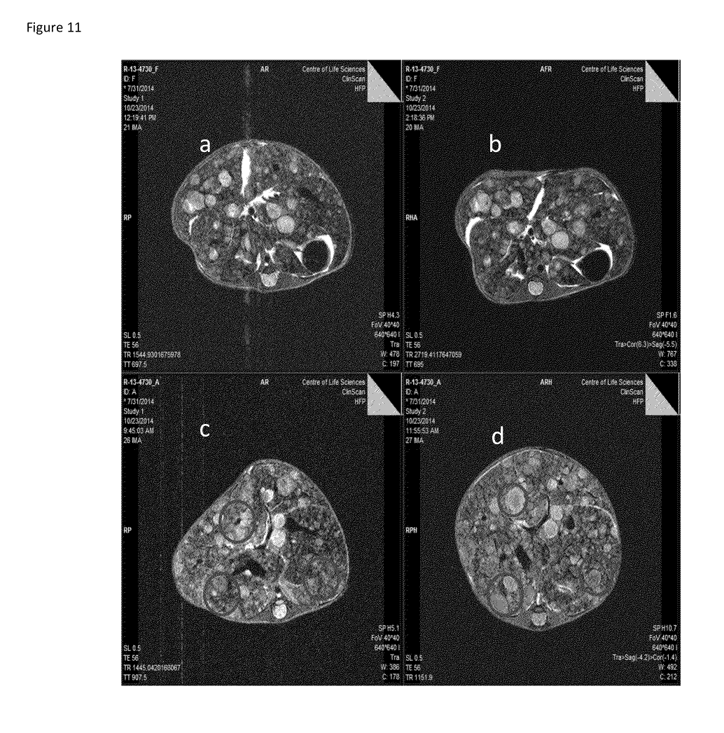

The T₁/T₂ weighted images were taken after 1 hour injection. The results are shown in FIGS. 10 and 11 respectively. In case of the T1-weighted images, tumour could be observed only in panel (d) after ND-DOTA . . . Mn injection (FIG. 10). In case of the T2-weighted images, multiple tumours could be observed in both panels (c) and (d) but were more obvious in panel (d) after ND-DOTA . . . Mn injection (FIG. 11).

These results illustrate that the complexes of the invention provide useful and effective dual contrast agents.

### EXAMPLE 5

### Comparative \Toxicity Studies

THLE-2 cells are immortalized normal hepatocytes that are commonly used for in vitro liver toxicity studies. These were treated with MnCl₂(10 μM), the ND-MN complexes as described in Examples 2 (ND-Mn) and 3 (ND-NH₂—Mn) above (Mn 10 μM equivalent) for 24 hours. Following treatment, cells were analysed for growth/inhibition/viability by MTT assay.

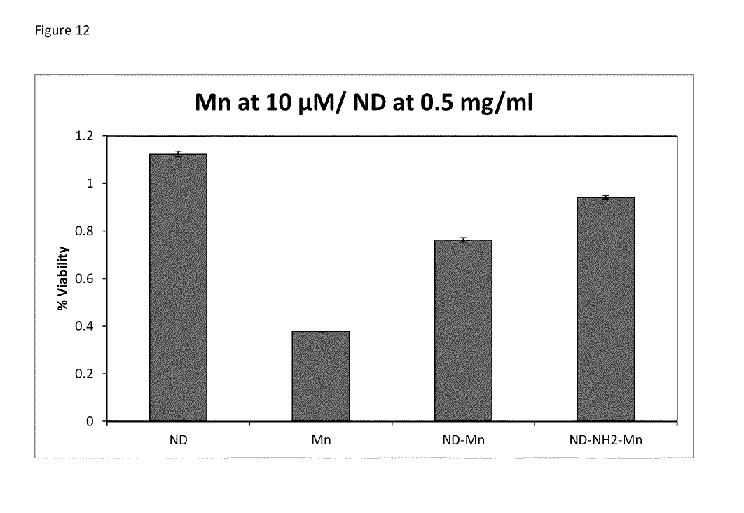

The results, normalized to untreated controls are shown in FIG. 12. These results show that ND-Mn complexes are less toxic to THLE-2 cells than MnCl₂.

Furthermore, non-human primate studies have been carried out for Maximum Tolerable Dose (MTD) of the ND-MN complexes, and have found No Observable Adverse Effect Level (NOAEL).

1. A reagent comprising a nanodiamond particle linked to a paramagnetic ion for use as a contrast agent in magnetic resonance (MR) imaging in which T2-weighted magnetic images are obtained.

2. The reagent according to claim 1 for use in magnetic resonance (MR) imaging, in which both T1 and T2 weighted magnetic images are obtained.

3. The reagent according to claim 1 wherein the paramagnetic ion is Mn²⁺.

4. A dual-mode contrast agent (DMCA) comprising nanodiamond linked to a paramagnetic ion selected from Mn²⁺, Eu³⁺, Tm³⁺, Yb³⁺ and Fe³⁺.

5. The dual-mode contrast agent according to claim 4 wherein the paramagnetic ion is Mn²⁺.

6. The dual-mode contrast agent according to claim 4 wherein the nanodiamond particles are less than 10 nm in diameter.

7. The dual-mode contrast agent according to claim 4 wherein the paramagnetic ion is linked to the nanodiamond particle by way of an organic linking group.

8. The dual-mode contrast agent according to claim 7 wherein the organic linking group comprises a chelating moiety which sequesters the paramagnetic ion.

9. The dual-mode contrast agent according to claim 7 wherein the organic linking group comprises surface activating moieties which are linked directly to the surface of the diamond nanoparticle.

10. A method for preparing a dual-mode contrast agent according to claim 4, said method comprising:
(A) reacting a paramagnetic ion with a nanodiamond particle which carries a chelating group that forms a conjugate with said paramagnetic ion; or
(B) reacting a nanodiamond particle with a chelate comprising said paramagnetic ion wherein said chelate is able to link to said nanodiamond particle or a functional group present on the surface of the nanodiamond particle; and
recovering the dual mode contrast agent from reaction mixtures formed.

11. The method according to claim 10 which comprises reacting the paramagnetic ion with a nanodiamond particle which carries a chelating group that forms a conjugate with said paramagnetic ion.

12. The method according to claim 11 wherein the nanodiamond particle which carries a chelating group is obtained by reacting a chelating agent with a nanodiamond particle having an activated surface.

13. The method according to claim 12 wherein the nanodiamond particle having an activated surface is obtained by contacting a nanodiamond particle with a surface activating agent.

14. The method according to claim 13 wherein the surface activating agent is an aminoalkyl or aminoaryl-alkoxysilane.

15. A pharmaceutical composition comprising a reagent according to claim 1 in combination with a pharmaceutically acceptable carrier.

16. The pharmaceutical composition according to claim 15 which comprises or more other active agents selected from one or more other imaging reagents or pharmaceutically active agents.

17. A method for obtaining a magnetic resonance image of a cell, tissue, organ, or subject, said method comprising administering to said cell, tissue, organ, or subject, a reagent comprising a nanodiamond particle linked to a paramagnetic ion, and subjecting said cell, tissue, organ, or subject to an MRI procedure in which a T2-weighted magnetic image is obtained.

18. The method according to claim 17 in which both a T1 and T2 determination is made.

19. The method according to claim 17 wherein the paramagnetic ion is Mn²⁺.

20-21. (canceled)

22. A pharmaceutical composition comprising a reagent according to claim 4 in combination with a pharmaceutically acceptable carrier.

23. A kit comprising a reagent comprising a reagent according to claim 4 together with an additional element required in MRI procedures.
